(19) United States
(12) Patent Application Publication
(10) Pub. No.: US 2002/0052571 A1
(43) Pub. Date: May 2, 2002

(54) Artificial kidney and methods of using same

(76) Inventor: Frank A. Fazio, Southboro, MA (US)

Correspondence Address: Jenifer E. Haeckl, Esq., Mirick, O'Connell,, DeMallie & Lougee, LLP, 100 Front Street, Worcester, MA, 01608, US

(21) Appl. No.: 09/952,617

(22) Filed: Sep. 13, 2001

Related U.S. Application Data
(60) Provisional application No. 60/232,126, filed on Sep. 13, 2000.
(60) Provisional application No. 60/232,125, filed on Sep. 13, 2000.

(51) Int. Cl. A61M 37/00

(52) U.S. Cl. 604/5.01

(57) ABSTRACT

An implantable, or compact, extracorporeal, artificial kidney that generally comprises: at least one blood inlet conduit, at least one microfluidic filter, a support for said filter, at least one blood outlet conduit and at least one blood solute outlet conduit, and methods for using the kidney.

### FIELD OF THE INVENTION

This invention relates to an artificial kidney that separates waste products and excess water from the blood stream and methods for using the artificial kidney as an implanted or external device.

### BACKGROUND OF THE INVENTION

It is long recognized that a need exists for an artificial kidney that can enhance the chances of survival for patients in End Stage Renal Disease (ESRD) or kidney failure. At present, there are approximately 320,000 people in the United States alone who are suffering from ESRD. The current medical options include kidney transplant or dialysis treatment. Approximately 35,000 of these patients are considered critical and are on a kidney transplant waiting list. Current estimates indiciate that approximately 24,000 of these patients will not receive a kidney transplant from either a healthy live donor or a cadaver, due to organ shortages. The remainder of the 320,000 ESRD patients will continue to be dependent on hemo- or peritoneal dialysis for survival.

### SUMMARY OF THE INVENTION

It is therefore a primary object of this invention to provide an artificial kidney to separate waste products and excess water from the blood stream that is adapted as an alternative or complete replacement of current techniques of hemodialysis, peritoneal dialysis or kidney transplant.

It is a further object of this invention to provide an implantable and/or external artificial kidney and methods for using the artificial kidneys.

A preferred embodiment of the compact artificial kidney of the invention is adapted to be worn or otherwise externally positioned near or on a patient's body and generally comprises: at least one blood inlet conduit, at least one microfluidic filtering means, a means for supporting said filtering means, at least one blood outlet conduit and at least one blood solute outlet conduit. The artificial kidney may further comprise at least one dialysate inlet conduit, wherein said blood and said dialysate pass through said microfluidic filtering means by laminar flow. The artificial kidney may still further comprise a manifold adapted to collect blood leaving said filtering means and to transport said collected blood to said blood outlet conduit and/or blood inlet and outlet cuffs adapted to facilitate communication and attachment between a patient's arterial system and said blood inlet conduit and between said blood outlet conduit and a patient's venous system, respectively. Another preferred embodiment of the artificial kidney of the invention is adapted to be implanted within a patient's body and comprises: at least one blood inlet conduit, at least one microfluidic filtering means, a means for supporting said filtering means, at least one blood outlet conduit and at least one blood solute outlet conduit. Similarly, the implantable artificial kidney may further comprise at least one dialysate inlet conduit, wherein said blood and said dialysate pass through said microfluidic filtering means by laminar flow. The microfluidic filtering means may be implanted within a patient's bladder and may further comprise a manifold adapted to collect blood leaving said filtering means and to transport said collected blood to said blood outlet conduit, wherein said blood solute outlet conduit preferably transports said blood solute to a patient's bladder for elimination through said patient's urinary tract.

Another preferred embodiment of artificial kidney that is adapted to be implanted within a patient's body, comprises: at least one blood inlet conduit, at least one ultra filtration matrix, a matrix cradle means for supporting said filtration matrix, and at least one blood outlet conduit, wherein at least one ultra filtration matrix is preferably configured as a coil. The matrix cradle preferably comprises a plurality of integral loops adapted to attach to an inner surface of a patient's bladder wall with a means for attachment, wherein said means for attachment may comprise a plurality of sutures. The artificial kidney may still further comprise blood inlet and outlet cuffs adapted to facilitate communication and attachment between a patient's arterial system and said blood inlet conduit and between said blood outlet conduit and a patient's venous system, respectively.

A preferred method of using an artificial kidney, generally comprises the steps of: providing an artificial kidney comprising at least one blood inlet conduit, at least one dialysate inlet conduit, at least one filtering means, a means for supporting said filtering means, and at least one blood outlet conduit; surgically implanting said artificial kidney inside a patient's body, including establishing fluid communication between the patient's arterial and venous systems and said blood inlet and outlet conduits, respectively; and periodically voiding blood solute from said filtering means through the patient's urinary tract, wherein said filtering means preferably employs microfluidic laminar flow. Similarly, the artificial kidney may be surgically implanted inside a patient's bladder.

### BRIEF DESCRIPTION OF THE DRAWINGS

Other objects, features and advantages will occur to those skilled in the art from the following description of the preferred embodiments and the accompanying drawings in which:

FIG. 1 is a schematic view of a preferred embodiment of the means for filtering used in the artificial kidney of the invention;

FIG. 2 is a schematic view of a preferred embodiment of the artificial kidney of the invention configured for external use;

FIG. 3 is a schematic view of another preferred embodiment of the artificial kidney of the invention configured for implantation in a patient's body; and

FIG. 4 is a schematic view of another preferred embodiment of the artificial kidney of the invention implanted into a bladder using another preferred embodiment of the means for filtering.

### DETAILED DESCRIPTION OF THE PREFERRED EMBODIMENTS AND METHODS

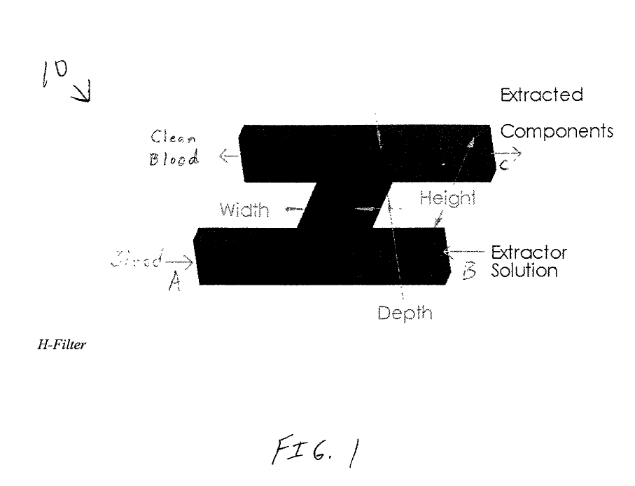

The invention features an artificial kidney that generally includes a blood inlet conduit, a means for filtering, a means for supporting the means for filtering, and blood outlet conduit. A preferred embodiment of the artificial kidney of the invention utilizes, in part, an H-filter design which, in turn, utilizes microfluidic technology. (E.g. Micronics Inc., Seattle, Wash.) This H-filter design is generally shown and referred to in FIG. 1 as filter means 10. Microfluidic technology provides a rapid separate mechanism based on diffusion. Microfluidic devices are based on low Reynolds number flow. The Reynolds number is the ratio of inertial forces to viscous forces. In a microfluidic flow environment, two dissimilar fluids employ laminar flow whereby the two streams flow side-by-side in a non-recirculating, non-mixing manner. The only exchange between the fluids takes place by diffusion of molecules from areas of high concentration to areas of low concentration.

By employing microfluidic, laminar flow, a microfluidic cassette performs the function of hemodialysis. An average person's kidney typically needs to extract about 18 g of urea in a 24 hour period from a volume of 5 liters of blood, assuming that the urea concentration is about 0.2-0.4 g/l of blood. Based on the average person and microfluidic, laminar flow rate, to achieve a comparable extraction efficiency, the filter cassette should have a volume of approximately 1×2×3 inches, not including hardware or ancillary components, to contain enough H-filters (e.g. about 200 layers of 5 mm wide H-filter channels). If extraction is desired to occur over a shorter period of time, then the volume of filter material may be increased accordingly.

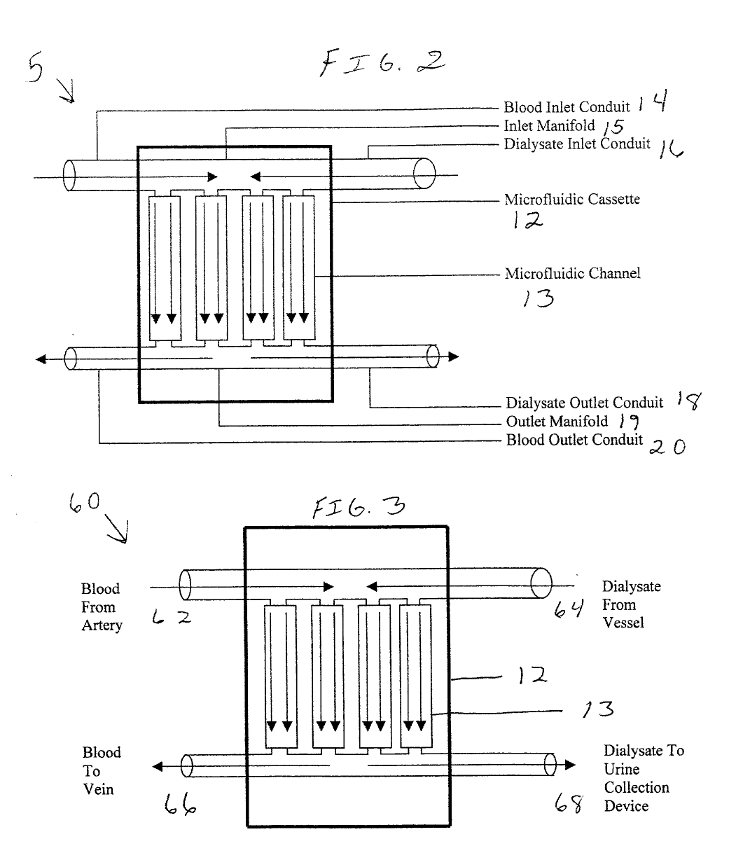

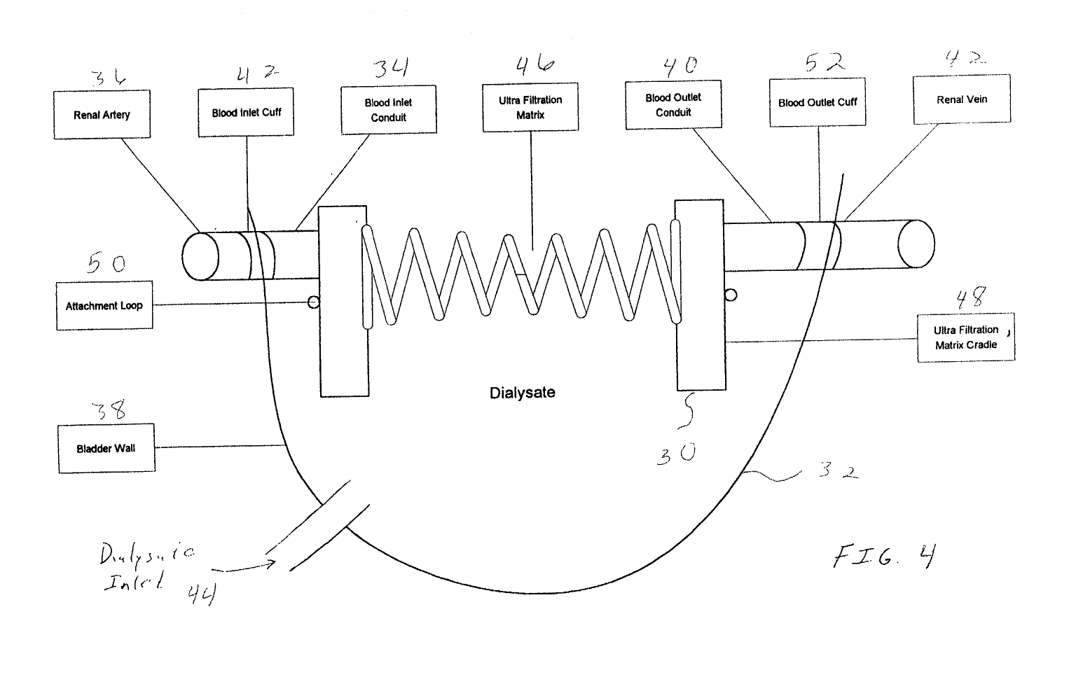

There are at least two preferred embodiments of the artificial kidney of the invention that utilize microfluidic technology, an external, or wearable, artificial kidney and an implantable artificial kidney. The external, or wearable, artificial kidney is preferably adapted to be worn or otherwise externally attached to a person, and yet, while still functionally connected to the person's renal system, can be positionally moved some distance away from the body. For example, it may be desirable to move the bulk of the external device to a relatively close location, such as a nightstand to make sleeping more comfortable. Both devices are adapted to utilize a general microfluidic cassette design shown in FIGS. 2 and 3 and referred to as cassette 12. The cassette comprises between 150 to 200 microfluidic channels 13 of filter 10. Blood from a patient's artery (arrow A) is introduced into the microfluidic cassette through each of the 200-microfluidic channels 13 along with dialysate (extractor solution arrow B). The two fluids flow together in a parallel, yet separate, laminar flow so that the fluids do not mix. As the fluids move through the channels, molecules diffuse from the blood to the dialysate. The table below, although not meant to be limiting in quantity or weight, indicates the most common blood solutes removed (extracted components arrow C), their respective molecular weights, and the typical mass removed per a twenty-four hour period.

| Component | Quantity | Mol Wt |
|---|---|---|
| Sodium | 4 g | 23 |
| Potassium | 2 g | 39.1 |
| Magnesium | .2 g | 24.3 |
| Calcium | .3 g | 40.1 |
| Iron | .2 mh | 55.85 |
| Ammonia | 1 g | 17.03 |
| H⁺ | Avg pH = 6 | 1 |
| Uric Acid | .8 g | 168.12 |
| Amino Acids | .15 g | Various |
| Hippuric Acid | .08 g | 179.2 |
| Chloride | 250 mEq | 35.4 |
| Bicarbonate | 50 mEq | 61 |
| Phosphate | 1.6 g | 97 |
| Inorganic Sulfate | 1.8 g | 98 |
| Organic Sulfate | .2 g | 98 |
| Urea | 18 g | 60 |
| Creatinine | .8 g | 113.12 |
| Peptides | .7 g | Various |

The composition of the dialysate is adapted to prevent the diffusion of solutes that should not be removed from the blood. Currently existing dialysate may be used with the device, including, but not necessarily limited to, hyper osmolar solutions. The resulting interaction between the blood and the hyper osmolar solution also causes excess water in the blood to also move from the blood into the dialysate during the hemodialysis process.

The external, wearable embodiment, shown generally and referred to in FIG. 2 as device 5, comprises a blood inlet conduit 14, an inlet manifold 15, a dialysate inlet conduit 16, a microfluidic cassette 12, a blood outlet conduit 18, an outlet manifold 19 and a dialysate outlet conduit 20. The blood inlet conduit is a catheter manufactured of materials demonstrated to have low thrombogenicity to whole blood. The blood inlet conduit has a standard luer type fitting for attachment to a catheter implanted into a patient's arterial system. The blood moves through the device using the patient's blood pressure or by an externally applied force. The dialysate inlet is a catheter with a standard luer fitting for attachment to the dialysate source. The dialysate is driven through the device by an external source. Both of the inlet conduits distribute their respective fluids to each and every channel in the microfluidic cassette. The blood and dialysate movement through the cassette and hemodialysis takes place as described above. The blood is then collected from each of the channels by a manifold and delivered to the blood outlet conduit. The blood outlet conduit is a catheter manufactured of materials demonstrated to have low thrombogenicity to whole blood. The blood outlet would have a standard luer type fitting for attachment to a catheter implanted in the patients venous system. The dialysate is collected from each of the channels by a manifold and delivered to the dialysate outlet conduit. The dialysate outlet conduit is a catheter manufactured so that it is compatible with current urinary collection systems. The waste dialysate is collected in a standard urinary waste collection system and disposed of through standard means.

The external or wearable artificial kidney is worn in a specially designed carrier that is worn on a belt around the patient's waist or in a vest. The dialysate reservoir is carried in the same belt or vest. The patient is required to change the dialysate reservoir several times per day. The waste dialysate is collected in a standard urinary collection device. The patient is required to change the urine collection system several times per day.

A preferred implantable embodiment of the artificial kidney utilizes the same microfluidic cassette used in the external artificial kidney and is generally shown and referred to in FIG. 3 as device 60. Device 60 is preferably implanted in a patient's bladder, however it may be implanted in any other suitable location within the body. The blood inlet conduit comprises a catheter surgically implanted into the patients arterial system. The blood inlet conduit distributes the arterial blood to the microfluidic channels 13. Blood may be similarly moved through the device using the patient's blood pressure or by an externally applied force. The dialysate inlet conduit 64 is a pressurized system that is accessed by the patient several times per day. The dialysate is distributed to each of the microfluidic channels. Hemodialysis takes place as the blood and dialysate move through the channels together. The blood is collected in a manifold and delivered to the blood outlet conduit 66. The blood outlet conduit is a catheter surgically implanted in the patients venous system. The waste dialysate is collected from each of the microfluidic channels and delivered to the dialysate outlet conduit 68. This conduit is a catheter connecting the implantable artificial kidney and the patient's bladder. The waste dialysate is transferred from the implantable artificial kidney directly into the patient's bladder for elimination through the urinary tract. A pressurized dialysis reservoir is the mechanism preferably used to move the dialysate through the device and into the bladder.

Another preferred embodiment of the artificial kidney of the invention, shown in FIG. 4 surgically implanted within a patient's bladder and generally referred to as device 30. Similarly, device 30 is preferably implanted in a patient's bladder, however it may be implanted in any suitable location in the body. The implanted device is connected to the arterial and venous systems of the patient. A dialysate is introduced into the bladder 32, and, at the appropriate time after introduction, is voided by the patient as is urine. The device thus requires only the periodic replacement of the dialysate for proper functioning.

All the embodiments of the artificial kidney of the invention are designed to restore the glomerular function of the patient's diseased kidney(s). The blood inlet conduit 34 of the implantable artificial kidney is attached to the arterial system of the patient at renal artery 36. The device is surgically inserted into the bladder and attached in a semi rigid fashion to the interior wall 38 of the bladder. The blood outlet conduit 40 of the implantable artificial kidney is attached to the venous system of the patient at renal vein 42. A transdermal device such as a catheter or drug delivery port 44 is used as a dialysate inlet. The catheter or port is attached through the wall of the bladder giving access to the interior of the bladder. A dialysate is then introduced into the bladder completely surrounding the implanted artificial kidney. As blood moves through the device, driven by the heart or by an externally applied force, waste and excess water moves from the blood, across the semi permeable membrane of the ultra filtration matrix, and into the dialysate. Waste and excess water are then voided from the patient as urine via the patient's urinary tract. The dialysate is then replaced through the transdermal device at prescribed intervals. The purified blood is introduced into the patient's venous system through the blood outlet conduit 40.

The blood inlet conduit 34 is connected to the renal artery of the living patient. The blood inlet conduit 34 preferably comprises the same semi-permeable material as the ultra filtration matrix, thus allowing the passage of the waste products and excess water through the walls of the conduit, or of an impermeable, implantable grade polymer suitable for the passage of blood through the conduit. The blood inlet conduit 34 preferably has an integral cuff 42 facilitating the attachment of the renal artery and allowing for attachment of the blood inlet conduit and renal artery junction through the wall of the bladder. The cuff may be constructed from Dacron® or another similar inert material and may be attached around the circumference of the inlet and outlet conduits to provide an area on the conduit suitable for passage through the bladder wall. The bladder wall articulates with the cuff. The bladder wall is sutured in a leak-proof manner to the cuff.

Device 30 is preferably arranged in a manner that increases the amount of conduit surface area forming the ultra filtration matrix 46. The conduit may be coiled, or arranged in vertical, horizontal or cylindrical matrixes. The ultra filtration matrix of the conduit serves as the primary vehicle for the exchange of waste and excess water from the blood to the dialysate. The blood is excised of waste and excess water by osmotic pressure as it moves through the conduit ultra filter.

The ultra filter is preferably supported by a bio-compatible polymer cradle 48 that adds rigidity to the ultra filtration matrix. The cradle may be formed in a manner that contains the semi-permeable ultra filtration unit without restricting the amount of surface area contacting the dialysate. The cradle preferably has integral loops 50 or portals that allow the assembly to be attached to the wall of the bladder.

The blood outlet conduit 40 is connected to the renal vein of the patient. The blood outlet conduit preferably comprises the same semi-permeable material as the ultra filtration matrix to allow the waste products and excess water to pass through the walls of the conduit, and/or may comprise an impermeable, implantable grade polymer suitable for the passage of blood through the conduit. The blood outlet conduit preferably has an integral cuff 52 that facilitates attachment to the renal vein and attachment of the blood outlet conduit and renal vein junction through the wall of the bladder.

The filtration matrix material is not necessarily limited to semi-permeable material, but may also comprise an ion exchange media, a biological filter, and/or a mechanical separation/filtration device.

Although specific features of the invention are shown in some drawings and not others, this is for convenience only as some feature may be combined with any or all of the other features in accordance with the invention.

Other embodiments will occur to those skilled in the art and are within the following claims:

What is claimed is:

1. An artificial kidney, adapted to be worn externally proximate to a patient's body, comprising, at least one blood inlet conduit, at least one microfluidic filtering means, a means for supporting said filtering means, at least one blood outlet conduit and at least one blood solute outlet conduit.

2. The artificial kidney of claim 1, further comprising at least one dialysate inlet conduit.

3. The artificial kidney of claim 2, wherein said blood and said dialysate pass through said microfluidic filtering means by laminar flow.

4. The artificial kidney of claim 1, further comprising a manifold adapted to collect blood leaving said filtering means and to transport said collected blood to said blood outlet conduit.

5. The artificial kidney of claim 1, further comprising blood inlet and outlet cuffs adapted to facilitate communication and attachment between a patient's arterial system and said blood inlet conduit and between said blood outlet conduit and a patient's venous system, respectively.

6. An artificial kidney, adapted to be implanted within a patient's body, comprising, at least one blood inlet conduit, at least one microfluidic filtering means, a means for supporting said filtering means, at least one blood outlet conduit and at least one blood solute outlet conduit.

7. The artificial kidney of claim 6, further comprising at least one dialysate inlet conduit.

8. The artificial kidney of claim 7, wherein said blood and said dialysate pass through said microfluidic filtering means by laminar flow.

9. The artificial kidney of claim 6, wherein at least said microfluidic filtering means is implanted within a patient's bladder

10. The artificial kidney of claim 6, further comprising a manifold adapted to collect blood leaving said filtering means and to transport said collected blood to said blood outlet conduit.

11. The artificial kidney of claim 6, wherein said blood solute outlet conduit transports said blood solute to a patient's bladder for elimination through said patient's urinary tract.

12. An artificial kidney, adapted to be implanted within a patient's body, comprising, at least one blood inlet conduit, at least one ultra filtration matrix, a matrix cradle means for supporting said filtration matrix, and at least one blood outlet conduit.

13. The artificial kidney of claim 12, wherein at least one ultra filtration matrix is configured as a coil.

14. The artificial kidney of claim 12, wherein said matrix cradle comprising a plurality of integral loops adapted to attach to an inner surface of a patient's bladder wall with a means for attachment.

15. The artificial kidney of claim 14, wherein said means for attachment comprises a plurality of sutures.

16. The artificial kidney of claim 12, further comprising blood inlet and outlet cuffs adapted to facilitate communication and attachment between a patient's arterial system and said blood inlet conduit and between said blood outlet conduit and a patient's venous system, respectively.

17. A method of using an artificial kidney, comprising the steps of:
providing an artificial kidney comprising at least one blood inlet conduit, at least one dialysate inlet conduit, at least one filtering means, a means for supporting said filtering means, and at least one blood outlet conduit;
surgically implanting said artificial kidney inside a patient's body, including establishing fluid communication between the patient's arterial and venous systems and said blood inlet and outlet conduits, respectively; and
periodically voiding blood solute from said filtering means through the patient's urinary tract.

18. The method of claim 17, wherein said filtering means employs microfluidic laminar flow.

19. The method of claim 17, wherein said artificial kidney is surgically implanted inside a patient's bladder.

20. A compact artificial kidney, comprising, at least one blood inlet conduit, at least one microfluidic, laminar flow filtering means, a means for supporting said filtering means, at least one blood outlet conduit and at least one blood solute outlet conduit.
